(19) United States
(12) Patent Application Publication
(10) Pub. No.: US 2015/0026889 A1
(43) Pub. Date: Jan. 29, 2015

(54) SURGICAL TABLES

(71) Applicant: Stille AB, Solna (SE)

(72) Inventors: Johannes Roselius, Stockholm (SE); Ralph Tamm, Solna (SE)

(21) Appl. No.: 14/512,553

(22) Filed: Oct. 13, 2014

Related U.S. Application Data
(63) Continuation of application No. PCT/SE2013/050403, filed on Apr. 15, 2013.

(30) Foreign Application Priority Data
Apr. 13, 2012 (SE) 1250369-4

(51) Int. Cl. A61B 6/04; A61G 13/04

(52) U.S. Cl. CPC A61B 6/0442; A61G 13/04; A61G 2210/50
USPC 5/601; 5/620

(57) ABSTRACT

The present invention relates to a surgical tables and table tops with reduced thickness and improved radiolucency of X-ray radiation. The invention also relates to surgical table arrangement adapted for the improved table tops.

### FIELD OF INVENTION

The present invention relates to surgical tables and table tops with reduced thickness and improved radiolucency of X-ray radiation. The invention also relates to surgical table arrangement adapted for the improved table tops.

### BACKGROUND OF THE INVENTION

European Patent Specification EP 1267723 describes a surgical table arrangement with a table top with radiolucent characteristics. This arrangement admits improved capacity to adjusting and locating the table top with patient in relation to an imaging equipment, such as a C-arm of an X-ray imaging equipment. Fine adjustments of the imaging thereby become possible without relocations of the table or the imaging equipment.

There have been many technical efforts made to improve surgical table tops to admit less absorption of X-rays and to avoid density variations and yet retain sufficient rigidity, stability other necessary mechanical properties and excellent image quality from the detector. A reduction in radiation is both desirable for the professional operators of X-ray equipment and the examined patients, while it gives a longer life to the expensive source of X-ray.

For this reason, table tops are suggested to be produced from composite materials that are free from or have minimized metal parts in their constructions. It is well established that carbon fibers are useful in such constructions due to favorably low X-ray absorption. Examples of such table tops are found in GB 2057830; WO 2007/106877; CA 1136777 and JP 2246936. A conventional way of manufacturing such table tops is to select a core member of a radiolucent thermoplastic resin with a suitable shape, covering with it carbon fiber and reinforcing plastics and producing a layered construction. JP 2005046182 describes such a top plate for patient mounting in a radiodiagnosis apparatus made from reinforced carbon fibers, without describing any X-ray absorption results.

Accordingly, there is still a need to further develop surgical table arrangement and provide table tops with still improved permeability for X-rays over a large surface area by a reduction in thickness and optimal use of composite forming materials and yet exhibiting mechanical characteristics.

It is also desirable to arrange a surgical table so the table top attached to a support in one end so that a remainder of the table top hangs freely. This design is particularly important when it comes to those requiring the use of for example a mobile C-arm. The table top may be movable along the longitudinal axis by means of two side rails running in slides at the support. The side rails are attached along the sides of the table top, either continuously or intermittently along the length of the rails. A problem experienced with these surgical tables is that the table top bends when a person lies on it, especially if the person is heavy. When the table top is bent, the side rails are also affected by the bending forces which make the rails to bend, too. This counteracts the movement of the table top since the slide means receiving the rail in a slidably manner during movement of the table top cannot receive the bent rail freely slidable and gets jammed. In order to overcome the risk of jamming the slides, articulated slides are used, but a resistance remains.

In summary, there is a need to combine improvements in more radiolucent table tops made thinner with supporting means in surgical table arrangements that admits reductions in radiation, especially from X-ray, while the mentioned problems are eliminated. The invention as described in the following parts represents technical solutions that improve surgical tables and examinations with radiation, especially for reducing radiation doses for patients subjected to endovascular surgical procedures.

### DESCRIPTION OF THE INVENTION

The present invention is in one aspect generally directed to table tops for surgical table arrangements with improvements in reduced X-ray absorption, methods of their manufacturing and an operable surgical table arrangement including such an improved table top, especially useful for endovascular surgical procedures by admitting reduction in radiation doses between 20 and 40%

The table tops according to the invention have significantly reduced thickness and an optimized employment of composite material, thereby admitting a reduction in X-ray attenuation and obtaining mechanically suitable characteristics. A surgical table arrangement according to the invention is adapted to connect the so improved table top and to support it so forces exerted from a load are suitably distributed and balanced.

The term “about” as used in the present context has the meaning that variations of 5% or less from a nominal value signifies borders defining the term about.

In one aspect, the present invention relates to a surgical table top comprising a core member made of an X-ray transparent thermoplastic foam material, and a composite material in a layer surrounding the core member, comprising from 50 to 60% (vol) of carbon fiber and a resin.

According to this aspect, the table top has an X-ray attenuation equivalence of from 0.4 to 0.6 mm Al (aluminum) at 100 kV; it has a thickness of less than 40 mm; and it tolerates a maximum load of 300 kg. According to this aspect, the surgical table top is produced with resin transfer moulding process, comprising arranging the core member and at least one sheet of carbon fiber on each side of the core member in a mould; sealing and heating the mould; injecting a pressurized resin forming system into mould until all carbon fiber voids are filled; and admitting the resin to cure at a curing temperature.

In this context, an X-ray attenuation equivalence is a comparative value related to the thickness of an aluminum sheet and is standard reference for technology estimating transparency of X-rays.

Further, in this context a composite material comprises essentially carbon fibers and a cured resin. For such a composite material comprising about 55% (vol) carbon fibers the remaining part is the resin. In this context “about” has the meaning of a variation not exceeding 1-2%. In this context a resin is a cured or crosslinked polymer resulting from a fluid resin forming system evenly distributed with the carbon fibers, while an essentially non-porous composite material is a material essentially free from entrapped air or any voids which may lead to variations in density and compromised imaging quality during surgery.

Also, in this context the term “tolerates a maximum load” has the meaning of explaining that up this value, the surgical table top is mechanically stable and will not be impacted by any load below the maximum load.

Also in the context of this aspect, resin transfer molding is a closed moulding process, wherein a pressurized liquid resin forming system is introduced in mould. For a reference, see Resin Transfer Moulding, K. Potter, 1997, Published by Chapman & Hall, 2-6 Boundry Row, London SE1 8NH, UK. By performing a resin transfer moulding process, the fibers are admitted an even and thorough contact and the mentioned essentially non-porous characteristics is accomplished.

In one aspect, the surgical table top has a core member made from a closed cellular polymethacrylimides (PMI) with a thickness of from 25 to 35 mm.

In one aspect, the surgical table top is produced with two carbon fiber sheets are arranged on each side of the core member with a combined thickness of the two sheets not exceeding 1 mm.

In one aspect, the two sheets of carbon fiber at each side of the core member have combined weight not exceeding 1000 g at a 60 vol % fiber level.

In one aspect, the layer of composite material is about 1 mm thick.

In one aspect, the composite material is essentially non-porous.

In one aspect, the resin forming system is a thermosetting epoxy resin.

In one aspect, the surgical table top has a vault with a radius exceeding 1000 mm.

In one example the radius is about 1250 mm and the vault has a height of about 47 mm.

In one aspect the surgical table top has composite material comprising about 55% (vol) carbon fiber and has an X-ray attenuation equivalence of 0.4 mm Al (aluminum) at 100 kV.

Suitable materials for the core member are found among radiolucent thermoplastic foam materials such as closed cellular polymethacrylimides (PMI). Especially suitable materials are found among materials having the trademark Rohacell® from Evonik Rohm GmbH, such as Rohacell® IG and Rohacell® (DIG-F. A particularly suitable such material is Rohacell® IG71.

It is also preferred that the core member is vaulted in order support rigidifying the table top and its profile. The core member has a suitable thickness of between about 25 to about 35mm. An especially suitable core member has a thickness of about 30 mm and is made of Rohacell® IG71.

Preferably, the two carbon fiber sheets used to make the core member surrounding composite material have a controlled thickness and do preferably not together exceed about 1 mm thickness. Suitable such carbon fiber sheets comprise a first carbon fiber sheet with a thickness of 0.28 mm and a weight of 280 g/m², and a second carbon fiber sheet has a thickness 0.60 mm and a weight of 620 g/m². HS carbon fibers are considered a suitable grade.

In one aspect, the invention relates to a surgical table adapted to and having an easy and exact positioning of the table top as described and produced in the previous section of the description, preferably giving a feeling of floating when moving the table top.

According to a one aspect, the surgical table comprises the table top arranged movable for receiving a patient thereon, wherein the table top is supported by a support in one end thereof. Two rails are connected to each side of the table top in the direction of the longitudinal axis of the table top. The rails slide along the length axis through slide means, and there is at least one slide means on each side of the support for receiving the rail. The rails are connected to the table top at one or two connection points on each side.

In one aspect, the table top has a first connection point of one rail situated in the same end as the support and a second connection point situated in the other end of the rail.

In one aspect, the table top has a first connection point of one rail on one side aligned with a first connection point of the rail on the other side along a thought orthogonal axis to the length axis.

In one aspect, the table top is rotatably connected about the connection points which further decrease the bending forces on the rails originating from the table top. Further, the rotatable connection counteracts concentrations of bending forces on the table top and a more favorable distribution of forces when the table top is loaded and deflected. The advantages from the just recited embodiments contribute to the opportunity of reducing the thickness from excess composite material in the table top and thereby in improvements in radiolucency and the mentioned desirable associated improvements from reductions in radiation.

In one aspect, each of the first and/or second connection point(s) comprises a pin. In another embodiment the pin may be rotatable inside a receiving holder or recess.

In one aspect, the first connection point is in the same end as the support and the second connection point is separated from the first connection point with about 0.50 to 1.50 m.

In one aspect, the distance between the connection points is long enough to reduce the bending forces originating from the loaded table top but situated in each end of the rail.

In one aspect, each rail is connected to the table top at one connection point only, and preferably at a first end portion of the table top which is supported by the support.

With the described surgical table arrangement, the previously described table top obtains a maximum deflection of less than about 30 mm at the maximum load of 300 kg. The arrangement generally admits or supports the reduction of the table top thickness and the associated optimization of the composite material, thereby further contributing to the improvements in radiolucency and advantages in imaging and reductions in radiation.

The present invention provides a solution that counteracts the establishment of the bending forces from the bent table top to the rails, thereby facilitating the sliding movement of the rails through the slide means. This allows the movements in the length axis to float in a very smooth way.

### DETAILED DESCRIPTION OF THE INVENTION

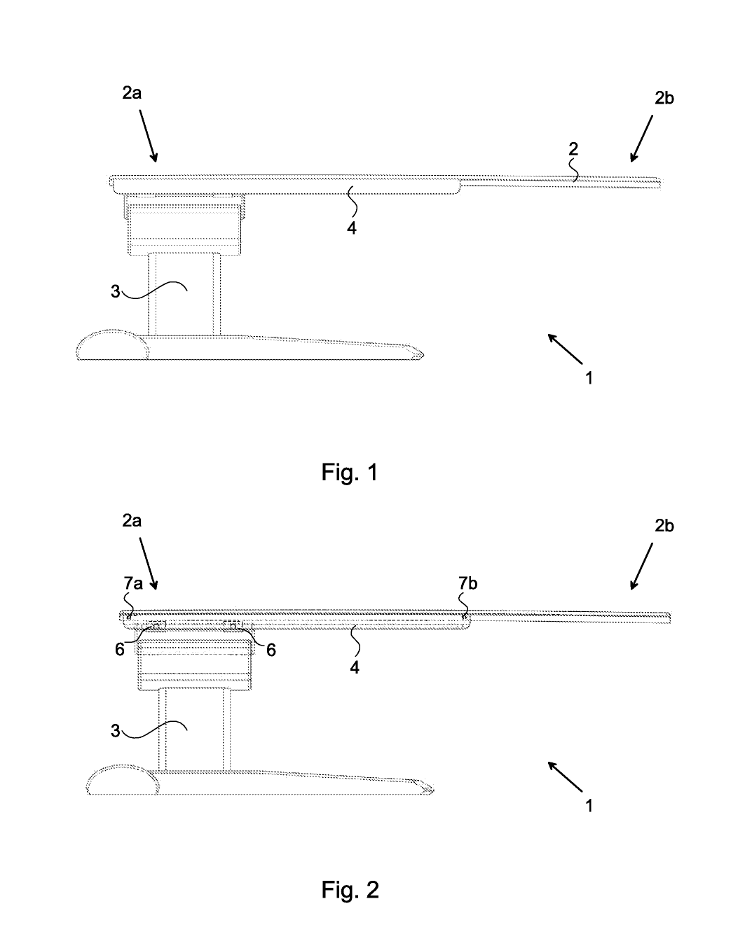

FIG. 1 shows a side-view the table top in surgical table arrangement in an unloaded position.

FIG. 2 show a sectional side view of the surgical table arrangement of FIG. 1.

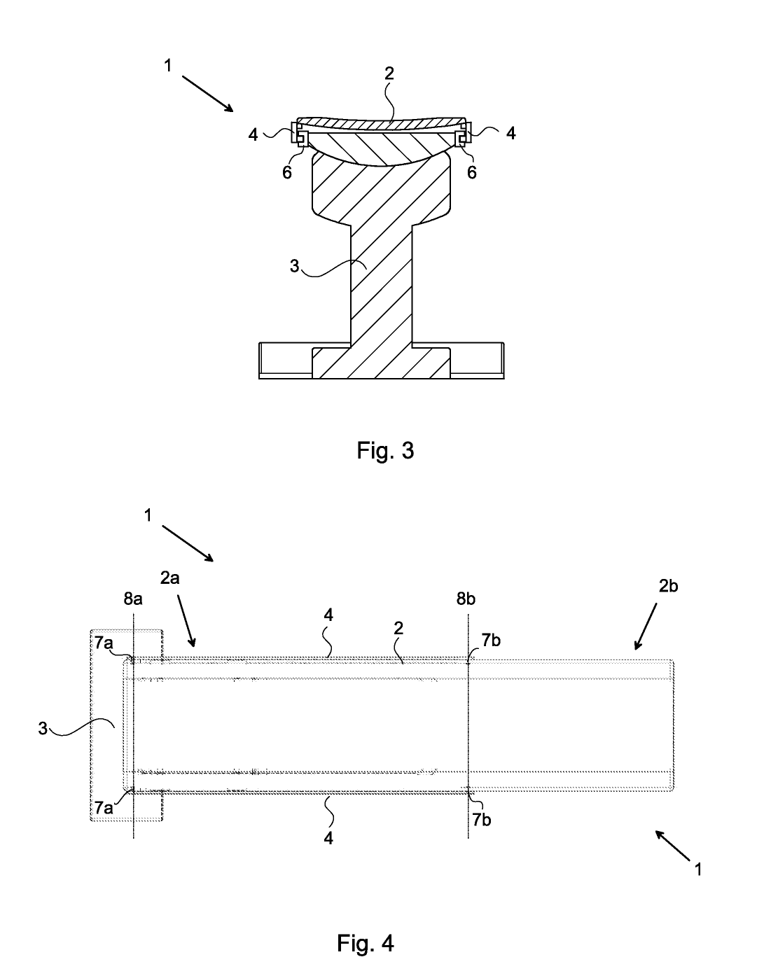

FIG. 3 shows an end view of the surgical table arrangement.

FIG. 4 shows a top view of the surgical table arrangement.

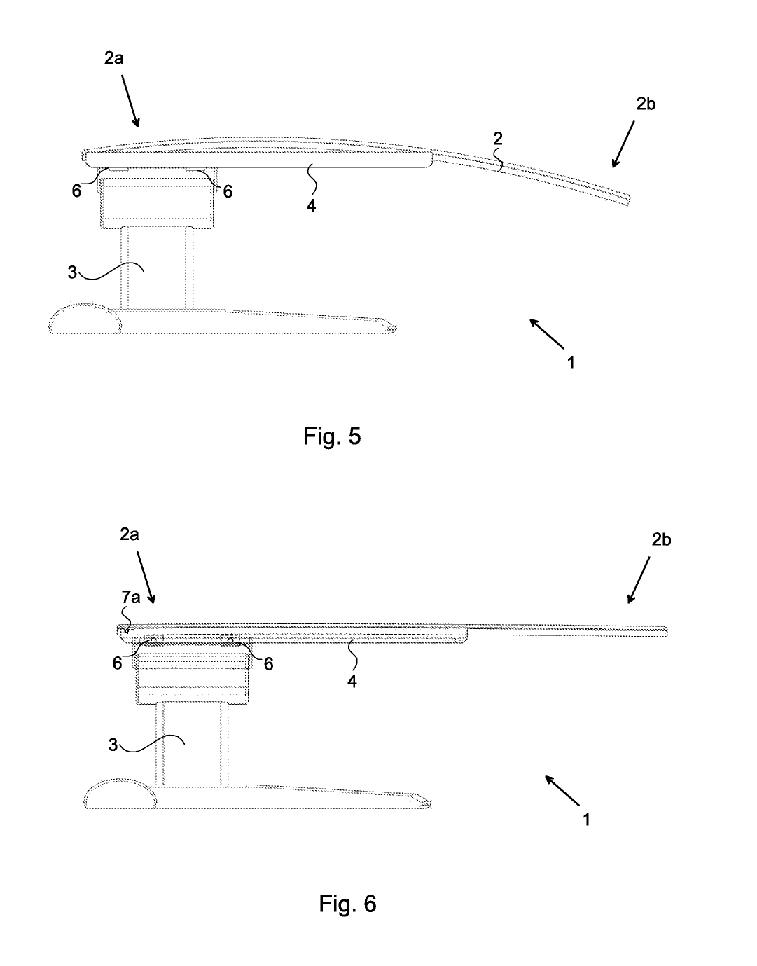

FIG. 5 shows a side view of the surgical table arrangement shown in FIGS. 1-4 but in a loaded position.

FIG. 6 shows a sectional side view of a second embodiment of a surgical table similar to that of FIGS. 1-5 but wherein the table top is attached in only one point at each side of the table top.

Before the invention is disclosed and described in detail, it is to be understood that this invention is not limited to particular materials or configurations disclosed herein as such configurations and materials may vary. It is also to be understood that the terminology employed herein is used for the purpose of describing particular embodiments only and is not intended to be limiting since the scope of the present invention is limited only by the appended claims.

The present invention will now be described in more detail hereafter with reference to the accompanying figures, in which embodiments of the invention are shown. This invention may, however, be embodied in many different forms and should not be construed as limited to the embodiments set forth herein.

For the production of improved table tops according to the present invention a core member of made from Rohacell IG71 is selected with a thickness of 30 mm. The table top to be produced is depicted with reference (2) in FIGS. 1 to 6 as part of a surgical table arranged so as to move the table top and to counteract bending forces exerted on the table top when exposed to loads. Other dimensions and contour features of the table top are described in the previously mentioned EP 1267723 or demonstrated on the homepage of Stine AB (http ://www.stille.se/stille_eng/Produkter/patientpositionering/documents/imagiObrochure2011.pdf).

The core is provided with vault of a few centimeters to increase comfort and improve stiffness of resulting table top which thereby will obtain a generally cup-shaped form as is shown in the cross-sectional view of FIG. 3.

In order to produce a table top that meet the requirements of being sufficiently rigid and tolerate high bending loads the core member is subjected to a reinforcing process so as to form a layered rigid structure with the core and a composite material. For this purpose the preformed core member is placed in a mould between double layers of sheet-formed carbon fibers, i.e. two sheets on either side of the core member. The mould is generally adapted to align with the shape of the core member.

A first carbon fiber sheet has a thickness of 0.28 mm and a weight of 280 g/m², and a second carbon fiber sheet has a thickness 0.60 mm and a weight of 620 g/m². The sheets are of the HS carbon fiber quality. A first and a second such sheet is placed in the bottom of the mold and a first and a second such sheet placed on top of the core member, whereupon the mould is sealed. The moulding is performed with the resin transfer moulding. Resin transfer moulding is an established closed moulding process for making fiber composite materials useful with a wide variety of carbon fibers and resin systems.

Resin Transfer Molding (RTM) is a method of fabricating high-tech composite structures. RTM uses a closed mold commonly made of aluminum.

In the manufacturing of the table tops of the present invention, the mentioned carbon fiber “layup” sheets are placed together with the carefully milled-to-shape mentioned sandwich core of Rohacell IG71, as well as metallic inserts for the fasteners into the open mold. The mold is then closed, sealed, and heated, whereafter the resin is injected into the mold to impregnate the fiber layup. The resin is a thermoset type resin which contains resin, hardener and possibly a catalyst in a liquid mix which is able to inject in the mould inlets impregnate the fiber completely until all voids are completely filled with the liquid resin. The injection pressure is carefully controlled between 2-15 Bar along a carefully selected time-pressure curve. The thermoset type resin is an epoxy material is found among conventional commercially available products and is required to thoroughly wet the carbon fibers in order avoid any density variations and other irregularities.

Because all the chemical active components is already in the liquid thermoset mix in the injection phase the thermoset mix will start to crosslink through chemical reaction, initiated by the temperature and when necessary a catalyst. The mold is then held at a temperature sufficient to cure the resin until the resin is solid and the mould can be opened. The heating temperature to start up and complete the chemical crosslink reaction is commonly between +50° and +150° C.

A table top product produced as described has a thickness of about 32 mm, i.e. an about 30 mm thick core member and an about 1 mm thick layer of composite material surrounding the core member. The composite material includes about 55% (vol) of carbon fiber and thereby an improvement in reduced content of epoxy material which has importance for the desired X-ray attenuation. A so produced table top has a length of 2300 mm a width of 550 mm, tolerates maximum load of 300 kg without any mechanical defects. It has and has the important feature of a low X-ray attenuation (X-ray radiolucency) of 0.4 mm aluminum (Al) attenuation equivalence at 100 kV (when tested with a RTI Barracuda w. R100B detector according to IEC 6061-1-3:1994 at MEDIEL AB's facility in Molndal, Sweden). The vault has a radius of 1250 mm and a height of 47 mm (c.f. FIG. 3). The deflection in an arrangement described below and depicted in FIGS. 1 to 5 is 27 mm (as calculated with FEM).

FIGS. 1 and 2 show a side view of a surgical table 1, wherein in FIG. 1 some of the interior parts are also shown. The surgical table 1 comprises a table top 2 produced in accordance with process described above, which in these figures are shown without the influence of a patient's weight. The table top 2 has two end portions, namely a first end portion 2a which is supported by a support 3, and a second end portion 2b which is free, i.e., is not directly supported by the support. A rail 4 is connected to the table top 2 on each side thereof in one or two connection points 7a, 7b, see FIG. 2. This embodiment has two connection points; the first connection point 7a is in the same end of the rail 4 as the supported first end portion 2a of the table top 2, and the second connection point is in the other end of the rail 4 closer to the free second end portion 2b of the table top 2. The rails 4 comprise connection means in the form of pre made holes so that the rails 4 are connectable to the table top 2 by means of pins, as will be described below. The table top 2 comprises in turn the corresponding predetermined connection means that the connection to the rails 4 requires. This embodiment show a rail 4 with pre-made holes connected to the bed via pins. The connection may be fixed or rotatable round an axle. The rails 4 extend partly along the length of the table top 2. The surgical table 1 has in this embodiment two slide means 6 on each side of the support 3 for slidingly receiving the rail 4 along the length of the table top. At least one clamping or locking element (not shown) is adapted to block the sliding movement of the rails 4 thereby locking the table top 2 in a desired longitudinal position. The rails 4 on each side of the table top 2 are less influenced by the bending of the table top 2 due to free rotation around pins in the connection points 7.

FIG. 3 shows the surgical table 1 in a cross sectional view from the free end 2b of the table top 2. The somewhat U-shaped table top 2 is supported by the support 3 and connected to the rails 4 on each side. The rails 4 on each side of the table top 2 are slidably movable in the slide means 6 along the length of the table top. The rails 4 on each side are connected to the table top 2 via pins inserted into the connection points 7 from the external side received of an adapted recess or nut. The pins may be rotatable 360 degrees inside respective connection point 7 further decreasing the bend forces on the rails 4.

FIG. 4 shows a top view of the table top 2 having the supported end portion 2a on the left side and the free end portion 2b on the right side of the drawing. The rails 4 extend only partly along the length of the table top 2. One connection point 7a supports one end of a respective rail 4, and the other connection point 7b supports the other end of a respective rail 4. The second connection point 7b of each rail 4 is at a distance long enough to move the bending force further away from the support 3. This solution moves the second connection point 7b closer to the free end portion 2b of the table top, which means that a smaller length is prone to bend because of a heavy patient. The connection points 7a, 7b in the first and second ends of each of the rails 4 are provided at the same distance along the table top 2 on the two sides thereof. This means that the connection points 7a at the supported first end portion 2a of the table top 2 are aligned with each other along a virtual first axis 8a, which means that the table top 2 is rotatable about this first axis 8a. Similarly, the connection points 7b at the free second end portion 2b of the table top 2 are aligned with each other along a virtual second axis 8b, which means that the table top 2 is rotatable also about this second axis 8a.

FIG. 5 shows a side view of the same surgical table 1 as in the previous figures, but in a somewhat exaggerated view when a heavy patient is positioned on the table top. Due to the heavy weight distributed along the table top 2, a bending effect will form and the table top 2 will bend down as shown in the drawing. The bending mechanism of the table top 2 can be compared to the trampoline principle, where the free end over the water bends due to a person's weight. When the table top 2 is bent by the bend forces, the bend forces will be transmitted to the rails 4. The rails 4 will be less influenced by the bending forces due to that they are connected to the table top in only a first and a second connection point 7a, 7b situated in its both ends.

However, since each rail 4 is connected to the table top in only two connecting points 7a, 7b on each side, the rails 4 are almost unaffected by the bent table top 2, which in turn enables the rails 4 to slide smoothly in the slide means 6 without jamming. This allows the movements in the length axis to float in a very smooth way.

FIG. 6 shows a second embodiment of a surgical table wherein the table top 2 is connected to the rail 4 in only the first connection point 7a on each side. The table top 2 lies on a pin that may be joined in the corresponding connection points 7a. In all other aspects this second embodiment is identical to the first embodiment described above with reference to FIGS. 1-5 and reference to these figures are made for understanding this second embodiment.

Finally, the present invention provides a solution that counteracts the establishment of the bending forces from the bent table top 2 to the rails 4, thereby facilitating the sliding movement of the rails through the slide means 6. This allows the movements in the length axis to float in a very smooth way.

Other features and uses of the invention and their associated advantages will be evident to a person skilled in the art upon reading the description and the examples. Thus, the connections means provided to interconnect the table top and the rails can be any suitable means allowing the table top to take on a bent shape when the weight of a patient exerts a pressure on the top of the table top. For example, the table top can be provided with brackets on the underside thereof adapted to receive a shaft extending perpendicularly to the longitudinal direction of the table top, wherein the shaft is attached to the two rails.

1. A surgical table top, comprising:
a core member made of an X-ray transparent thermoplastic foam material, and a composite material in a layer surrounding the core member, comprising from 50 to 60% (vol) of carbon fiber and a resin, characterized in that it:
has an X-ray attenuation equivalence of from 0.4 to 0.6 mm Al (aluminum) at 100 kV;
has a thickness of less than 40 mm;
tolerates a maximum load of 300 kg; and in that
it is produced with a resin transfer moulding process, comprising arranging the core member and at least one sheet of carbon fiber on each side of the core member in a mould; sealing and heating the mould; injecting a pressurized resin forming system into mould until all carbon fiber voids are filled; and admitting the resin to cure at a curing temperature.

2. A surgical table top according to claim 1, wherein the core member is made from a closed cellular polymethacrylimides (PMI) with a thickness of from 25 to 35 mm.

3. A surgical table top according to claim 1 or 2, wherein two carbon fiber sheets are arranged on each side of the core member with a combined thickness of the two sheets not exceeding 1 mm.

4. A surgical table top according to claim 3, wherein the two sheets of carbon fiber at each side of the core member have combined weight not exceeding 1000 g at a 60 vol % fiber level.

5. A surgical table top according to claim 1, wherein the layer of composite material is about 1 mm thick.

6. A surgical table top according to claim 1, wherein the composite material is essentially non-porous.

7. A surgical table top according to claim 1, wherein the resin forming system is a thermosetting epoxy resin.

8. A surgical table top according to claim 1, having a vault with a radius exceeding 1000 mm.

9. A surgical table top according to claim 1 with a composite material comprising about 55% (vol) carbon fiber with an X-ray attenuation equivalence of 0.4 mm Al (aluminum) at 100 kV.

10. A surgical table having a table top for receiving a patient thereon according to claim 1, a support for supporting the table top, two rails connected to the table top on each side in the direction of the longitudinal axis of the table top, and at least one slide means on each side of the support for slidingly receiving the rail along the longitudinal axis, characterized in that the rails are connected to the table top at one or two connection points on each side of the table top.

11. The surgical table according to claim 10, wherein a first connection point of one rail is situated in the same end as the support and a second connection point is situated in the other end of the rail.

12. The surgical table according to claim 10, wherein a first connection point of one rail on one side is aligned with a first connection point of the rail on the other side along a thought orthogonal axis to the length axis.

13. The surgical table according to claim 12, wherein the table top (2) is rotatable connected around the connection points.

14. The surgical table according to claim 12, wherein each of the first and/or second connection points comprises a pin.

15. The surgical table according to claim 10, wherein the first connection point is in the same end as the support and wherein the second connection point is separated from the first connection point with about 0.50 to 1.50 m.

16. The surgical table according to claim 10, wherein each rail is connected to the table top at one connection point only.

17. The surgical table according to claim 10, wherein each rail is connected to the table top at a first end portion thereof which is supported by the support.

18. The surgical table according to claim 10, wherein the table top has a deflection of less than 30 mm at the maximum load of 300 kg.
